(19) United States
(12) Patent Application Publication
(10) Pub. No.: US 2006/0210984 A1
(43) Pub. Date: Sep. 21, 2006

(54) Use of nucleic acid mimics for internal reference and calibration in a flow cell microarray binding assay

(76) Inventor: Jeremy Lambert, Worcester, MA (US)

Correspondence Address: Leon R Yankwich;Yankwich & Associates, 201 Broadway, Cambridge, MA, 02139, US

(21) Appl. No.: 10/548,229

(22) PCT Filed: Mar. 3, 2004
(86) PCT No.: PCT/US04/06479

§ 371 Date: Sep. 2, 2005

(30) Foreign Application Priority Data
Mar. 3, 2003 (US) 60451,468

(51) Int. Cl. C12Q 1/68; G01N 33/53; C12M 1/34

(52) U.S. Cl. 435/6; 435/7.1; 435/287.2

(57) ABSTRACT

The present application describes a method for normalizing for variations in signal intensity observed in a biomolecular binding assay carried out in a flow cell cartridge. Variations in signal intensity occur as a result of the effect of the surfaces of a flow cell cartridge on the laminar flow of reagent through the cartridge. In any individual reagent stream, fluid flows faster in the center of the stream and slower at the outer periphery of the stream due to contact of the reagent with the walls of the cartridge, creating a parabolic fluid flow profile. The present invention describes a method for normalizing or calibrating out the differences in intensity observed in different regions of interest on a single chip or similar reactions carried out in different cartridges, as a result of these differential fluid flow rates. Microarray chips having integrated calibration regions are also described.

### CROSS-REFERENCE TO RELATED APPLICATION

This application claims priority to U.S. Provisional Application No. 60/451,468 filed Mar. 3, 2003.

### FIELD OF THE INVENTION

The present invention is related to the field of high throughput proteomics and to equipment useful for the simultaneous analysis of up to thousands of biomolecular interactions occurring on the surface of a single microchip inserted in a flow cell cartridge. In particular, the present invention provides materials and methods for normalizing or calibrating for variations in signal intensity of binding reactions on a microarray chip due to variations in reagent flow rate over the surface of the chip that occur as a result of the contact between the flow stream and the surfaces of the flow cell cartridge. The present invention also provides a method for normalizing or calibrating for differences in signal intensity observed with similar reactions performed on separate chips and/or in different flow cell cartridges.

### BACKGROUND OF THE INVENTION

Recent developments in the field of proteomics have made high throughput screening assays a fast and reliable method for analyzing up to thousands of biomolecular interactions in a single assay. A typical high throughput screening assay employs a sensor chip having biomolecular ligands immobilized thereon in an ordered array, a processing unit having liquid handling capabilities for flowing an analyte solution over the surface of the chip, an optical unit for detecting binding interactions between the analyte in solution and an immobilized ligand, and a computer for processing and analyzing the binding data. Selective interactions of the analyte with the immobilized ligand gives this technique specificity, and also enables analysis of interactions in complex mixtures.

A number of methods are available for detecting the binding interaction between a ligand and analyte at the surface of the chip. For example, surface plasmon resonance (SPR) is a technique used to measure the change in the resonance angle as a function of the increase in refractive index caused by the binding of an analyte to a ligand. In another method, the analyte may include a fluorophore or a ligand for a fluorophore whereby the level of fluorescence is used to detect the presence of bound analyte. However, any method known in the art may be used for detecting a molecular binding reaction including, but not limited to chemiluminescence, fluorescence, colorimetry, surface plasmon resonance, electroluminescence, radiation, and/or MALDI-TOF mass spectra. In a microarray scanning system, the area of interest is usually comprised of an array of discrete elements referred to as “pixels”. Each pixel is illuminated independently as it is being addressed by the scanning system.

For analysis of a binding interaction, the sensor chip having the immobilized ligand(s) is secured in an integrated microfluidic cartridge such as depicted in FIG. 1. The cartridge (1) consists of a series of fluid flow channels (not shown) connected to one or more reagent reservoirs (2) and serves to conduct the flow of reagent, e.g., buffer, analyte solution, etc., from one or more reservoirs to the surface of the microarray chip (8). After contact with the surface of the chip, separate channels transport the reagents to waste receptacles (7) within the cartridge. The flow of reagent from individual reservoirs to the chip is controlled by a rotatable valve (12) having one or more conduits that align on one side with the one or more flow channels leading from the reservoirs (2) and on the other side with the channel or channels leading to the surface of the chip (8). Flow of reagent through the channels is controlled by a vacuum or other pressurized pump which is connected to the cartridge.

Control of fluid movement through the microfluidic cartridges is particularly problematic because of the microscale nature of the device. Proper control of fluids through flow paths is a challenge, as microdimensions impart characteristics and behaviors that are not encountered in larger scale fluidic systems, due primarily to the greater influence of surface effects within the flow cell cartridge in a microscale environment. For example, one difficulty in a laminar flow assay system is that during pressure-induced flow of fluids through microchannels, non-uniform flow velocities are experienced in individual flow streams due in part to friction that exists at the interface of the reagent and the surfaces of the cartridge during fluid transport. For example, due to the effects of the walls of the channels, a faster fluid and material flow is observed in the center of a moving laminar fluid stream than on the periphery of the moving fluid stream, creating a parabolic fluid flow profile (see FIG. 8). This differential resistance to flow, particularly over the surface of a functionalized sensor chip, can lead to undesirable, nonuniform assay conditions at the chip surface, where experimental conditions with respect to analyte binding differ on the outer edges of the sensor chip (where fluid flow is slower), as compared to the conditions on the center portion of the chip (where fluid flow is faster). Also, even slight variations between the individual flow channels in any given cartridge will affect the velocity of the reagents contacting the surface of the sensor chip, depending on the particular channel.

Controlling the rates of fluid flow through microchannels and reducing the surface effects that a flow cell cartridge has on laminar flow of reagents can be complicated and costly given the microscale nature of any design parameters. For example, U.S. Pat. No. 6,592,821 describes the use of fluids varying in density to focus a particle stream to the center of a flow channel to achieve a more uniform flow rate within the stream. U.S. Pat. No. 5,690,895 discloses a method for causing the flow rates of the peripheral and central portion of a flow stream to be more uniform via the introduction of a “sheath liquid” that surrounds the sample flow stream as it passes through the flow cell. U.S. Pat. No. 6,637,463 discloses a complicated multi-channel microfluidic system that employs pressure differential in individual channels to control fluid movement. Other methods include engineering a sensor chip with raised columns or depressions to cause the flow of solution over the chip to favor a turbulent instead of laminar flow pattern. Another method for accounting for the differences in flow rate in a single flow stream would be to monitor the differences in flow rate via a flow meter built into the cartridge, however the costs of such a cartridge would make it prohibitively expensive for most users.

Currently, there is a need in the art for a fast, accurate, and cost effective method for talking into account the effects of differential fluid flow rates in a single fluid stream in a high throughput microassay that does not involve the introduction of extraneous fluids into the system or require the development of complex design features. More specifically, there is a need in the art for a fast, reliable method for calibrating or normalizing for signal intensity variations observed in binding reactions carried out on a single sensor chip caused by variations in fluid flow velocity over the surface the chip, or calibrating or normalizing for signal intensity variations observed as a result of variations in reagent flow velocity between replicate microassays carried out on different sensor chips or in different flow cell cartridges.

### SUMMARY OF THE INVENTION

To solve this problem, the present invention employs the use of nucleic acid molecules, preferably peptide nucleic acid (PNA) oligomers, as an internal calibration and reference indicator in a microarray binding assay performed in a flow cell environment.

Peptide nucleic acids (PNAs) are compounds that possess similar characteristics and properties to oligonucleotides, however they are structurally distinct. For example, in contrast to the deoxyribose phosphate backbone of oligonucleotides, the backbone of PNAs are more akin to a peptide than a sugar phosphodiester. Specifically, the PNA backbone is made up of repeating N-(2-aminoethyl)-glycine subunits linked by peptide bonds, and the bases (purines and pyrimidines) are linked to the backbone by methylene carbonyl linkages. Unlike DNA or other DNA analogs, PNAs do not contain any pentose sugar moieties or phosphate groups. Because of this variation from the deoxyribose backbone, these compounds were designated “peptide nucleic acids”.

In addition, due to the peptide backbone, PNAs are not recognized by either nucleases or proteases and as a result, unlike DNA and proteins, are resistant to enzymatic degradation and remain stable over a wide range of pH.

PNAs bind both DNA and RNA to form PNA/DNA or PNA/RNA duplexes. The PNA backbone is not charged and, as such, exhibits strong binding characteristics with DNA and RNA due to the lack of charge repulsion between the individual strands. Also, due to the neutral (uncharged) backbone of the PNAs, no salt is required to favor and/or stabilize the formation of PNA/DNA or PNA/RNA duplexes and therefore, the Tₘ of the resulting duplex is independent of ionic strength. In this way, the PNA/DNA duplex interaction offers a further advantage over DNA/DNA duplex interactions which are highly dependent on ionic strength. In addition, homopyrimidine PNAs have been shown to bind complementary DNA or RNA forming (PNA)₂/DNA or RNA triplexes of high thermal stability (see, Egholm et al., Science, 254: 1497 (1991); Egholm et al., J. Am. Chen. Soc., 114: 1895 (1992); Egholm et al., J. Am. Chem. Soc., 114: 9677 (1992)).

Because of their properties, PNAs are known to be useful for a number of different applications. Since PNAs have stronger binding and greater specificity than oligonucleotides, they are used as probes in cloning, blotting procedures, and in applications such as fluorescence in situ hybridization (FISH). PNAs have further been used to detect point mutations in PCR-based assays (PCR clamping).

Protein microarrays have recently been described for the simultaneous detection of multiple antigens in a single assay (Haab et al, Genome Biology, 2(2): 4-13 (2001)). The use of small spots of capture antibodies or affinity capture molecules spatially segregated in a one or two-dimensional array format provides a means for analyzing hundreds or thousands of samples in a relatively small area, e.g., a microscope slide. However, the adoption of such protein arrays to high-throughput methods has been limited by the need for highly skilled technicians and expensive liquid-handling robots. (See, Schweitzer et al, Nature Biotech., 20: 359-365 (2002).)

Recently, a device for automated immunological and biochemical analysis has been described in which the entire assay can be performed within an integrated cartridge containing internal reagent reservoirs and a flow cell sensor for interfacing with an analytical detection device (see, PCT/US01/28692). The cartridge design provides an ideal platform for automated microarray binding assays (see FIG. 1).

The invention described herein provides a fast, reliable, and accurate method for calibrating or normalizing for uncontrollable variations in the signal intensity generated by molecular binding reactions taking place on the surface of a microarray chip as a result of the nonuniform flow rate of a laminar fluid stream in a flow cell cartridge. Specifically, it is known that the flow rate of a fluid stream through a microchannel is faster in the center of the stream and slower at the outer periphery of the stream, due to contact of the laminar fluid stream with, and the resulting friction from, the surfaces of the microchannel, in particular the walls of the channel. As demonstrated herein, this differential in flow rate causes a “false” variation in chemiluminescence intensity between molecular binding reactions taking place on different sections of a single chip.

The invention described herein provides a method for accounting for variations in fluorescence intensity that result from these variations in flow rate, and thereby improves the accuracy of results obtained from a qualitative or quantitative-type microassay via analysis of the binding reactions of designed nucleic acids, preferably peptide nucleic acids (PNAs), which are advantageously spotted at predetermined locations onto the surface of the microchip and included as part of the assay reaction. In particular, a homologous population of peptide nucleic acids are immobilized or “spotted” onto a microarray chip at one or more predetermined locations. Preferably, at least two or more spots of PNAs are arranged in a contiguous row or, more preferably, a contiguous column on the surface of the microarray chip. More preferably, the at least two or more spots of these PNAs are arranged in a column that is perpendicular to the flow of fluid across the surface of the microchip. Preferably, a first population of PNA oligos is spotted on a section of the microchip that is closer to the walls of the cartridge and a second population of PNA oligos is spotted closer to the center of the microchip, i.e., farther from the walls of the cartridge. According to this arrangement, and as described above, the first population of PNA oligos spotted close to the walls of the cartridge will be exposed to a portion of the reagent stream that is flowing slower than the portion of the reagent stream contacting the second PNA population spotted at the center of the chip. Most preferably, the microchip includes at least three populations of PNA oligos spotted in a line perpendicular to the flow of reagent and arranged such that one population of PNA oligos is spotted on a section of the microchip that is close to one wall of the cartridge and a second PNA population is spotted on a section of the microchip that is closer to the opposite wall of the cartridge from where the first PNA population is located and a third PNA population is spotted near the center of the microarray chip, i.e., farthest from either wall of the cartridge. According to this physical arrangement, the spots positioned by the wall of the cartridge will each be exposed to a portion of the reagent stream that is flowing at a slower rate than the center portion of the reagent stream.

The microarray chip may be advantageously contained in an integrated cartridge system such as described in PCT/US01/28692 and shown in FIG. 1. The preferred cartridge includes a number of individual reagent reservoirs for storing buffer or sample to be transported to the surface of the microchip. According to the present invention, at least one reservoir of the cartridge will include a population of nucleic acids, for example peptide nucleic acids, that are complementary to at least one of the nucleic acid spots immobilized on the chip as described above. The number of unique nucleic acid sequences or oligos used in the assay is preferably at least equal to the number of reagent reservoirs within the cartridge required to perform any particular assay. For example, if a particular assay requires the use of four cartridge reservoirs, for example for wash buffer, fluorescent detection ligand, etc., each reservoir preferably includes a unique population of nucleic acids that are different from the nucleic acid population of any of the other three reservoirs, i.e., the nucleic acids from one reservoir are not complementary (do not have affinity for) to any of the nucleic acids from any of the other reservoirs, and each reservoir nucleic acid is complementary (has a high binding affinity for) at least one nucleic acid population immobilized on the microchip. It should be noted that any population of nucleic acids in any reservoir may have more than one corresponding complementary nucleic acid spot immobilized on the microchip. However, it is also contemplated that any one reservoir may include more than one homologous population of nucleic acids as long as the resulting heterologous population of nucleic acids in each reservoir are noncomplementary (do not bind to each other) or the nucleic acid populations in any of the other reservoirs. Also, it will be understood by one skilled in the art that not every reservoir used in a binding assay according to the present invention will require a population of nucleic acid calibration molecules.

The unique population of nucleic acid oligomers included in each of the separate reagent mixtures are such that they are complementary to at least one of the individual populations of nucleic acid sequences spotted onto the surface of the microassay chip. As each reagent containing at least one unique, i.e., homogenous population of nucleic acids, is pumped through the flow cell cartridge and flows across the surface of the microchip, the nucleic acids contact the “capture array” of immobilized complementary nucleic acids on the surface of the chip to form a duplex on the chip. Such duplexes may be detectable by various detection means well known in the art. Thus, by detecting hybridization of complementary nucleic acids at nucleic acid calibration reaction spots on the chip, the flow of the reagent solution and the exposure of the immobilized spots to the reagent are confirmed. Preferably, the present method is employed to account for variations in signal intensity caused by localized variations in flow rate of reagents across the surface of the chip or variations in signal intensity between replicate assays carried out on more than one chip and/or in separate flow cell cartridges.

In a preferred embodiment, the binding reactions on the surface of the chip are designed such that the high and low average pixel intensity range of all of the analyte reaction spots on a chip fall within the high and low average pixel intensity range produced by the nucleic acid calibration reaction spots after hybridization with their complementary calibration molecule.

In a particularly preferred embodiment, a unique population of homogenous nucleic acids is deposited on the chip in a column of at least 4 individual spots spanning the surface of the chip and positioned so as to be perpendicular to the flow of reagent across the surface of the chip. (See, e.g., FIG. 2.) The specificity of the duplexes formed by each of the paired capture and detection nucleic acid sequences is controlled so that the cross-reactivity between non-complementary sequences is minimized, assuring that the signal produced at a given calibration reaction spot is produced by the hybridization of only the “detection” sequence in the reagent that is complementary to the immobilized “capture” sequence that makes up the calibration reaction spot (to the extent of the efficiency of the synthesis of the oligomers).

According to the present invention, a microarray binding assay is performed under conditions of laminar flow of reagent across the surface of the chip, and a comparison of the intensity of replicate calibration reaction spots, immobilized in a pattern that is perpendicular to the direction of reagent flow across the surface of the chip, is used to correct for variations in signal intensity observed at the analyte reaction spots resulting from localized variations in flow rate across the surface of the chip due to surface tension created by contact of the flowing reagent with the internal surfaces of the flow cell cartridge, in particular the walls of the cartridge. For example, as seen in FIG. 3, reagent flow rates are slower along the outer edges of the microassay chip in close proximity to the walls of the cartridge, presumably due to the surface effect or “drag” the walls of the cartridge have on flowing reagent. As seen in FIG. 3, this surface effect results in a (deceptively) higher intensity of the spots on the chip that are physically located closer to the cartridge walls due to the higher analyte concentration and longer period of time that the slower moving analyte in the solution is able to maintain contact with its complementary immobilized ligand. As seen in FIG. 3, the intensity of the spots immobilized near the center of the chip are uniformly of lower intensity than the spots on the outer edges of the chip due to the lower concentration and lower contact time between the faster moving analyte and its complementary immobilized ligand. As seen in FIG. 5, by following the methods of the present application, these variations in chemiluminescence intensity due to differential flow rates in a single reagent stream are essentially normalized or “calibrated out” of the reaction. (Compare FIGS. 3, 4 and 5.)

In a particularly preferred embodiment, the present invention is directed to a novel method for the normalization or automatic referencing of molecular binding reactions on the surface of a biosensor microarray chip. According to this method, a microarray flow cell cartridge such as depicted in FIG. 1 is provided with a microarray chip having at least one analyte reaction spot and at least one, preferably at least two, and more preferably, at least three, unique calibration reaction spots deposited thereon. Each analyte reaction spot comprises a plurality of analyte capture ligands specific for a particular analyte. The analyte capture ligands and analyte can be any biomolecules having the ability to form a binding complex or otherwise having an affinity for each other in a quantitative or qualitative manner. For example, such ligand/analyte pairs contemplated by the present invention include, but are not limited to, antibody/antigen, biotin/streptavidin, sense DNA/antisense DNA, enzyme/substrate, etc.

Each calibration reaction spot immobilized on the microchip comprises a unique population of homologous calibration capture ligands for a calibration molecule that is different from the analyte. Examples of calibration capture ligands and calibration molecules suitable for use in the present invention include any biomolecules having a measurable binding affinity. For example, the calibration capture ligand and calibration molecule may each represent one-half of a complementary pair of peptide nucleic acids (PNAs) with a high binding affinity for the formation of a PNA/PNA duplex. Other examples of suitable calibration capture ligands and calibration molecules suitable for use in the present invention include complementary DNA molecules having a high affinity for the formation of DNA/DNA duplexes, combinations of complementary DNA or RNA plus PNA molecules for the formation of DNA/PNA or RNA/PNA duplexes, or mixtures of complementary RNA molecules for the formation of RNA/RNA duplexes.

According to the present invention, each of the one or more reservoirs of the flow cell cartridge that will include a fluid reagent for use in a particular microassay may include at least one unique homogenous population of calibration molecules dispersed in the reagent. By “unique” it is meant that the nucleic acid sequence of the calibration molecule in any given reservoir is different from the nucleic acid sequence of any calibration molecule in any of the other reservoirs, so as to prevent the unwanted binding/interaction of calibration molecules from different reservoirs during the running of the assay and, more importantly, to provide a method for calibrating or normalizing reactions carried out with reagents from each reservoir, whether the reagent contains an analyte or is simply a wash buffer or other reagent without analyte. It should also be noted that any reservoir reagent may include more than one homologous population of nucleic acid molecules again, as long as each reservoir has its own unique population of nucleic acid molecules and as long as the different populations in each reservoir do not interact or have very low, preferably zero, binding affinity for each other.

Each of the reservoirs is connected to a fluid conduit for conducting the reagents, wash buffer, analyte, etc., from the reservoirs to the flow cell of the cartridge, and thence across the surface of the microarray chip, causing the reagent to flow across the chip in such a manner as to contact the analyte capture spot or spots and also the calibration molecule's complementary calibration capture spot or spots immobilized on the chip.

Following an optional wash step, the chip is analyzed for the presence of calibration molecules from each reservoir bound to the corresponding calibration reaction spots, the presence of one or more calibration molecules bound to a calibration reaction spot confirming that contact between said analyte and said analyte capture ligand has taken place and/or contact between said analyte detection ligand and said analyte has taken place.

In another embodiment, this method is also suitable for calibrating or normalizing for differences observed between similar microarray assays performed in different flow cell cartridges or variations between similar binding reactions performed on different, i.e., separate, microassay sensor chips. In addition to the surface effects that exist in a flow cell cartridge as described above, slight manufacturing differences between (otherwise identical) cartridges can also affect the rate of laminar flow from one cartridge to the next. Therefore, as described in more detail below, the present invention is also suitable as a fast, accurate, and reliable method for accounting for variations in microassay binding results that occur with similar reactions carried out in two or more flow cell cartridges.

The present invention also contemplates a microassay chip functionalized with at least one analyte reaction spot, and at least one, and preferably at least two homologous calibration reaction spots arranged in a line (column) perpendicular to the flow of reagent across the surface of the chip, with said at least one analyte reaction spot being arranged in a line (row) with at least one of the calibration reaction spots such that the analyte reaction spot and the calibration reaction spot are parallel with the flow of reagent across the surface of the chip. In a more preferred embodiment, the microassay chip of the present invention will include a plurality of calibration reaction spots arranged in a series of at least one and preferably at least two or more columns, each column comprised of a homologous population of calibration reaction spots, each calibration reaction spot comprised of preferably peptide nucleic acids, and each of said columns being comprised of spots of a different population of nucleic acid molecules, preferably peptide nucleic acids.

In another embodiment, the present invention also contemplates a prepackaged kit comprising a flow cell cartridge having at least one reagent solution with a calibration reaction molecule dispersed therein and a sensor chip having at least one calibration reaction spot immobilized thereon, said calibration reaction spot being comprised of immobilized ligands complementary to the calibration molecule in the reagent.

Definitions

As used herein, the term “analyte reaction spot” or “analyte capture spot” refers to an individual homogenous population of biomolecules immobilized (“spotted”) at at least one discrete location on a sensor chip, said biomolecules being capable of binding or hybridizing with a binding partner that is a ligand or analyte. Examples of biomolecules suitable for use in the present invention as analyte reaction spots or analyte capture spots, include, but are not limited to, antibodies, antibody fragments, antigens, nucleic acids, proteins, peptides, etc. Accordingly, the sensor chip of the present invention may include from one to several thousand individual analyte reaction spots, each comprised of a population of immobilized biomolecules specifically reactive with an analyte or binding partner. Each analyte reaction spot may be comprised of the same or different population of biomolecules as any other analyte reaction spot.

As used herein, the term “detection molecule” or “detection ligand” refers to any molecule that possesses the capability of binding to another molecule and can be analyzed to detect such binding. An example of a detection molecule would be any fluorophore capable of binding to another molecule and presenting a measurable fluorescent, chemiluminescent, colorimetric, SPR, etc., signal after such binding.

As used herein, the term “pixel” refers to the detectable signal created by the interaction of a detection molecule such as a fluorophore and its ligand. Preferably, according to the present invention, following completion of an assay, the intensity of each pixel on a reaction spot is measured and the intensity of each spot as a whole is measured as a function of the average pixel intensity of that spot.

As used herein, the term “calibration reaction spot” refers to a homologous population of nucleic acid molecules, immobilized at at least one discrete location on a sensor chip. Preferably, each microarray chip includes at least two calibration reaction spots. Examples of such nucleic acid molecules according to the present invention include, but are not limited to, peptide nucleic acids (PNA), DNA, RNA, and/or derivatives of such molecules. The nucleic acid molecules according to the present invention may be further functionalized according to methods well-known in the art, for the purposes of improving immobilization or binding affinities or for the detection of binding reactions.

As used herein, the term “analyte capture ligand” refers to the binding partner of an analyte. For example, if an analyte capture spot is comprised of a multiplicity of immobilized antibodies reactive with an antigen in a sample solution, the antigen may be regarded as the “analyte” and the reactive antibody may be referred to as the “analyte capture ligand”.

As used herein, the term “calibration capture ligand” refers to the complementary binding partner for the calibration nucleic acid reagent that is added to a reagent reservoir of the flow cell cartridge. For example, if a calibration capture spot (calibration reaction spot) is comprised of a homogenous population of peptide nucleic acids, the “calibration nucleic acid” will be a nucleic acid reagent (PNA, DNA, RNA) that is complementary to the immobilized PNA “calibration capture ligand” of the calibration spot. The nucleic acid reagent and the calibration capture ligand will be capable of hybridizing to form a duplex.

As used herein, the term “chip-specific calibration factor” refers to the calculated normalization or calibration of the binding reactions taking place on a single sensor chip as disclosed in the present application.

As used herein, the term “feature-specific calibration factor” refers to the calculated normalization or calibration of similar binding reactions carried out on at least two sensor chips or carried out in more than one flow cell cartridge as disclosed in the present application.

### BRIEF DESCRIPTION OF THE DRAWINGS

FIG. 1 is a schematic view of a cartridge-type flow cell adapted for microarray analysis. The spot density of the array (150 μm spots at 375 μm spacing), and shown in enlarged view in FIG. 1, is approximately 1600 spots/cm². The cartridge (1) is comprised in general of one or more reagent reservoirs (2) as described above, and a microarray chip compartment (3) with space for securing the microarray chip (8) therein. Said compartment (3) or flow cell is linked to the reagent reservoirs (2) via fluid channels (not shown) which transport reagent solutions from the reservoir to the flow cell (3) and thus over surface of the chip. The cartridge (1) also includes a sample injection port (4), an air pump inlet (5) for applying a vacuum or other pressure to the cartridge to drive the reagent fluids through the system, a waste receptacle (7) for collecting reagent after contact with the microarray chip (8), and a rotatable control valve (12), surrounded by a wall (6), said valve being suitable for directing reagent from specific reservoirs (2) to the microarray chip (8).

FIG. 2 shows an image of chemiluminescent signals produced on the surface of a microarray chip containing both capture antibody (specific for an analyte) and PNA calibration spots. Homologous PNA calibration reaction spots, comprised of a plurality of deposited capture PNAs, are arranged in four columns of homologous reaction spots (homologous referring to the fact that the same capture PNA sequence is used to form each of the four reaction spots in that column). One column of reaction spots for each of four capture PNAs (Capture 1, Capture 2, Capture 3, Capture 4) is shown. The variation in intensity between the capture antibody samples is a result of the different affinities of the matched antibody pairs used in the assay for each target cytokine (analyte). Table 1 lists the normalization factors for each calibration reaction spot, as well as the row-specific calibration factors for the array shown in FIG. 2.

FIG. 3 shows a plot of PNA calibration spot intensity for each of four rows from the microarray image shown in FIG. 2. A pattern of row-dependent signal intensity is apparent in the figure. The arrow designates the direction of laminar flow relative to the replicate calibration reaction spots.

FIG. 4 shows the intensity of the analyte capture spots for each of the ten unique capture antibodies spotted on the array. FIG. 4 also demonstrates the variation in intensity caused by localized variations in reagent flow rate over the surface of the microarray chip.

FIG. 5 shows the results of applying the row-specific calibration to the analyte capture spots. The variation in replicate spots is reduced as compared with FIG. 4.

FIG. 6 displays the average signal of calibration reaction spots from five replicate chips. Table 2 (infra) lists the feature-specific calibration factors as well as the chip-specific calibration factors for the five replicate chips.

FIG. 7 shows a comparison of average response from five replicate experiments with and without the use of PNA reference spots for normalization between arrays. The graph clearly shows the reduced assay variability between arrays when the PNA reference spots and normalization method disclosed herein are employed. Table 3 (infra) shows the uncalibrated and calibrated results using the PNA nucleotides according to the present invention for the five replicate experiments shown in FIG. 7.

FIG. 8 is a schematic diagram showing the parabolic profile of pressure-induced fluid flow over the surface of a microarray sensor chip (8). As illustrated in the diagram, the rate of flow of a fluid stream (11) over the surface of the microarray chip (8) in the direction of the arrows shown, is slower at the periphery of the stream, presumably due to contact of the moving fluid with the walls (10) of the flow cell cartridge. Also, as seen in the diagram, biomolecules immobilized in discrete spots (9) on the surface of the chip are exposed to non-uniform flow rates depending on their physical location on the surface of the chip (8). The present invention provides a method for calibrating or normalizing variations in assay results caused by this parabolic fluid flow and resulting differential in flow rates across different regions of the chip (8).

### DETAILED DESCRIPTION OF PREFERRED EMBODIMENTS

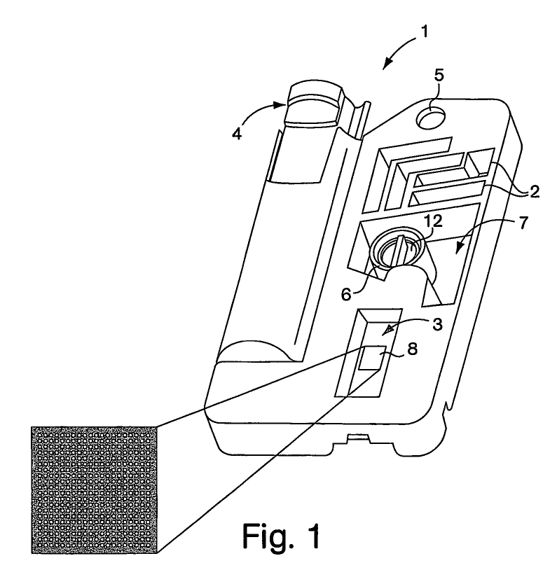

High throughput microassays are a valuable tool for the simultaneous analysis of up to thousands of separate biomolecular interactions on a single microchip in a flow cell cartridge. The present invention relates to methods for the calibration of binding reaction data performed in a high throughput microassay format, to account or compensate for undesirable variations in signal intensity of binding data caused by variations in reagent flow rate occurring in a flow cell cartridge. According to this method, homologous (or even heterologous) populations of biomolecules are immobilized at strategic locations onto the surface of a microchip as discrete “spots” in a two-dimensional array pattern. Each chip may contain up to thousands of these spots arranged in a regular pattern of rows and columns. In addition, each spot on a microchip may be specific for the same analyte, a different analyte, or any combination, with the number of different analytes being limited only by the number of spots that a single chip can physically accomodate. Once the microchip has been “functionalized” with these biomolecule spots, i.e., prepared for reacting with an analyte in solution, the chip is secured into the reaction chamber of a flowcell cartridge such as depicted in FIG. 1. As described more fully below, the cartridge includes reservoirs for holding reagents such as wash buffers or other solutions, including analyte solutions. The reagents in the reservoirs are then propelled, for example by vacuum or other pressure-based method, through channels within the cartridge that link the reservoirs to the flow cell reaction chamber where the microarray sensor chip is located. The solution from the reservoirs is then contacted or passed over the surface of the chip and then conducted via further tubing or channels to a waste collection receptacle included within the cartridge. A means for confirming that a binding reaction has taken place on the surface of the chip, such as use of a fluorescent ligand molecule that is specific for the analyte may be included in the solution of one of the reservoirs, or may be bound to the analyte in solution prior to contacting the chip, or may be contacted with the chip after the analyte binding reaction is complete. A separate fluorescent ligand is also used to detect binding of the calibration molecules. After the binding reaction is complete, the chemiluminescence intensity at each spot may be measured and quantitated by methods known in the art.

In particular, the present invention is directed to a method for normalizing or calibrating for the variations in fluorescence intensity protocols used to demonstrate or quantitate the amount of analyte present in a reagent in a microarray format, which variations are observed at different regions of a single microassay chip after completion of the assay. These protocols include, but are not limited to chemiluminescence, fluorescence, colorimetry, surface plasmon resonance, electroluminescence, radiation, and MALDI-TOF mass spectra.

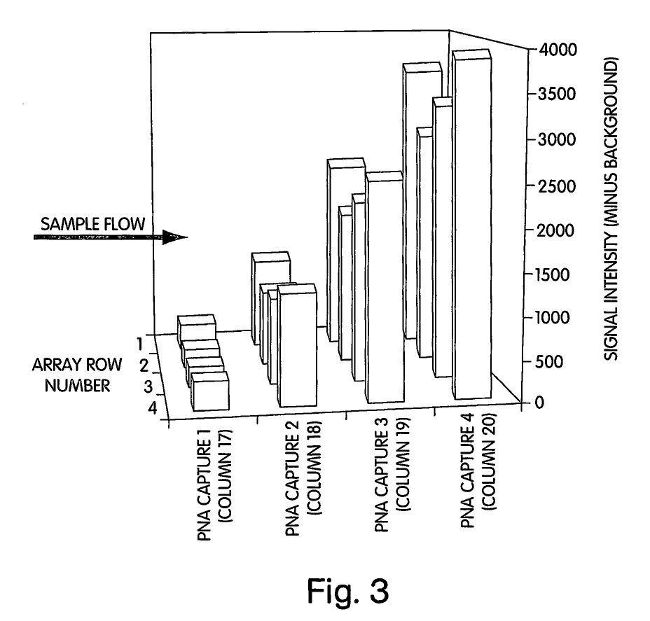

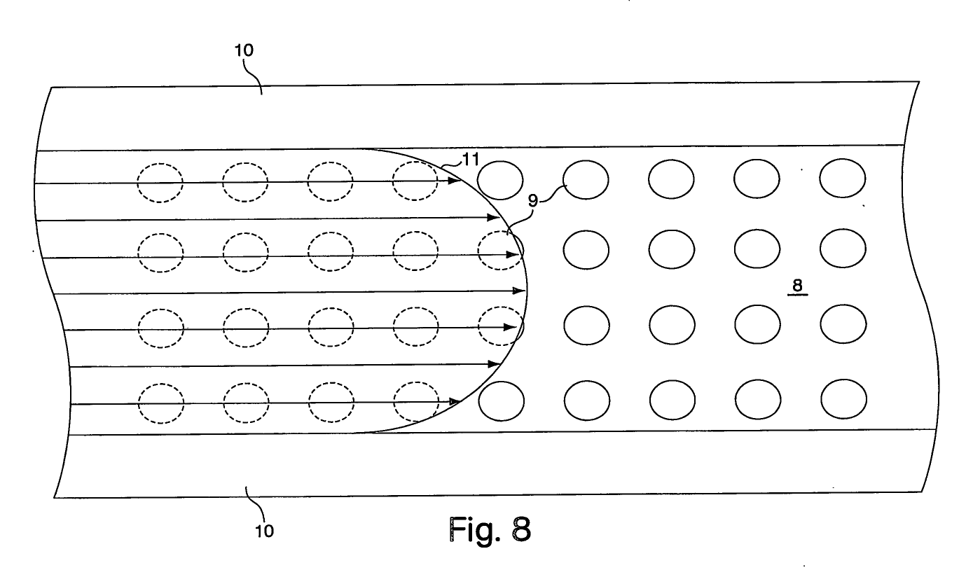

The observed variations are the result of localized differences in the physical conditions existing on the surface of the chip while the microassay is being performed. More specifically, these chemiluminescent intensity variations observed from one region to the next or from one spot to the next on the surface of a single chip are a function of the physical location of any particular reaction spot on the chip and are a direct result of the effects that the surfaces of a flowcell cartridge have on the laminar flow of a reagent solution within the cartridge. For example, it is known that friction caused by contact of a flowing reagent stream with the surfaces of a channel, in particular the walls of a flow cell, results in slight variations in the rate of fluid flow in any given region of the stream as a function of distance from the walls, and results in a parabolic fluid flow profile such as depicted in FIG. 8. Therefore, a particle flowing in a fluid stream will flow more slowly if it is crossing the cell at the outer periphery of the stream than if it were flowing at the center of the same stream. As a result of this differential in fluid flow velocities within a single stream, an analyte flowing at the outer periphery of the stream will move more slowly and be more concentrated (i.e., more analyte per unit area) than an analyte located closer to the center of the stream, which will be moving faster and at a lower concentration of analyte per unit area. This slower-moving analyte, or population of analytes, will be in contact with its immobilized ligand (on the sensor chip) for a longer period of time and at a higher concentration on any particular section of the chip that is closer to the walls of the cartridge than a population of analytes closer to the center of the moving stream and flowing faster and at a lower concentration. As a result, chemiluminescence intensity associated with reactions at the spots located on the outer edges of the chip, i.e., closer to the walls of the cartridge flow cell, are uniformly more intense than spots located near the center of the chip, farthest from the walls of the cartridge. (See, e.g., FIG. 3.) This variation in flow rate negatively affects the accuracy of any chemiluminescence data, as results will always appear to indicate that the analytes binding to the spots immobilized close to the edges of the chip are more concentrated and more abundant than analytes with complementary ligands immobilized closer to the center of the microchip. As stated above, it requires costly and complicated design parameters to eliminate these differential flow rates, especially in a microenvironment such as a cartridge flow cell.

The cartridge formatted flow cell for use in the present invention described herein may contain some or all of the necessary reagents and wash solutions required to run a complete series of quantitative or qualitative binding assays in a microarray format. The user loads (injects) the test sample into the cartridge and places the cartridge into an analytical instrument that has means to direct the fluid flow to the various areas of the cartridge, which areas are designed according to the assay to be performed. The cartridge design may allow for sample treatments such as heating, mixing, filtering, electroporation, cell lysis, or other chemical treatments prior to contact with the microarray sensor chip. The analytical instrument contains the components required for imaging the microarray and reporting the quantitative or qualitative results for each spot within the microarray. The detail blow-up of the microchip (8) inserted into the cartridge in FIG. 1 illustrates a sensor chip which is spotted with a microarray of bioreactive molecules, including spots comprised of calibration oligomers according to the present invention. Hundreds or even thousands of reactive areas can be spotted onto a single chip: Spot densities of 1600 spots per square centimeter or more are achievable.

The cartridge is designed so that all of the necessary reagents, wash buffers, etc., required to perform a sandwich-type microassay are pre-loaded into separate reagent reservoirs contained within the device. The analyte-containing sample or solution may be loaded into one or more of the reservoirs of the cartridge, or alternatively, may be added by injection into the cartridge via an injection port (4) shown in FIG. 1.

An array of affinity capture ligands (e.g., antibodies, aptamers, Fab fragments, scFv, nucleic acids, proteins, peptides etc.) including the reference and/or calibration “capture” oligomers, for example, PNA, DNA, or RNA oligomers as described herein are printed (immobilized) at predetermined locations onto a microarray sensor chip, i.e., as analyte reaction spots and calibration reaction spots, respectively. In a preferred embodiment of the present invention, the microchip includes at least one, preferably at least two, and most preferably at least three calibration reaction spots printed on the microchip in such a manner as to form a column of calibration reaction spots, said column being perpendicular to the direction the flow of sample and reagent solutions will take across the chip. In a particularly preferred embodiment of the present invention, on any single microchip, at least one calibration reaction spot is immobilized on a section of the microchip that is in close proximity to one wall of the cartridge chamber (3), at least another calibration reaction spot is immobilized on a section of the microchip that is in close proximity to the wall opposite the wall of the first calibration reaction spot, and at least one calibration reaction spot is immobilized close to or at the center of the chip. Again, in this arrangement, the at least three calibration reaction spots are arranged in a line (column) on the microchip so as to be perpendicular to the direction that solutions will flow across the chip.

A sensor chip printed with reactions spots suitable for the desired assay(s) is inserted into a flow cartridge such as depicted in FIG. 1, and a transparent window is assembled on top of the chip. A flow chamber is thus created between the window and the chip, e.g., by means of an adhesive gasket material of appropriate thickness to allow for flow of a wide range of biological samples that may contain analyte (e.g., whole blood, plasma, serum, cell lysate, tissue culture supernatant, purified proteins, peptides, nucleic acids, etc.), including the reference/calibration capture ligands as described herein.

Referring to FIG. 1, the flow chamber (3) of the cartridge (1) contains inlet and outlet ports (not shown) to allow for the flow of sample and reagent solutions conducted from the reservoirs across the surface of the sensor chip (8) and ultimately into a collection apparatus or waste reservoir (7). Each of the cartridge reservoirs (2) contain the reagents necessary to perform a quantitative or qualitative analysis of the sample, such as an analyte, introduced into the cartridge. In addition, one or more reservoirs include a population of single-stranded PNA, DNA, and/or RNA oligomer calibration capture ligand molecules complementary to one or more of the PNA, DNA, or RNA oligomer calibration ligands making up the calibration reaction spots immobilized on the microarray chip. Each reservoir, with a population of calibration nucleic acid molecules, includes a unique population of oligomers in that the nucleic acid sequence of the oligomers in any one reservoir is different, i.e., substantially nonhomologous, from the nucleic acid sequence of the population of oligomers in any other reservoir, so as to prevent binding interactions between the calibration molecules and, more importantly, to provide a unique nucleic acid molecule for normalizing or calibrating reactions from each reservoir as disclosed by the method of the present invention. This provides a calibration reference for each reservoir reagent (each mixed with a corresponding different detection molecule) to be contacted with the microarray chip. It should also be noted that each reservoir may contain a different type of calibration molecule depending on the nature of the molecules in any particular calibration reaction spot immobilized on the chip. In other words, calibration reagents of different types (PNAs vs. DNAs vs. RNAs) may be employed so long as there is a corresponding hybridization partner immobilized in a calibration reaction spot on the surface of the chip. For example, one reservoir of the cartridge may contain a population of PNA calibration nucleic acid molecules corresponding to (hybridizable with) one or more calibration reaction spots comprised of complementary PNA, DNA or RNA molecules on the chip, and another reservoir in the same cartridge may contain a population of DNA calibration nucleic acid molecules hybridizable with complementary capture nucleic acids on different calibration reaction spots immobilized on the chip. Each reservoir may also contain more than one population of homologous nucleic acid molecules as long as the resulting heterogenous population (of two or more homogenous populations of nucleic acid molecules) of molecules are noncomplementary, i.e., do not bind with each other in the same reservoir, thus confounding their ability to be bound at the reaction spots of the sensor chip.

The array feature of the cartridge design allows for detection of anywhere from one up to several hundred to several thousand different molecular targets (e.g., antigens, proteins, DNA, etc.) in a complex mixture. The multiplexed assay contemplated by microarray chips capable of capturing multiple targets is only advantageous if the assays are highly reproducible and if the individual real-time experiments/reactions accurately measure the concentration of multiple targets in a single assay. The level of assay automation built into the cartridge reduces the opportunity for assay variability caused by the user, since parameters such as the flow rate, time, reservoir, and temperature required for the assay may all be controlled by a computer running a pre-defined protocol. In addition, the calibration molecules used according to the present invention provide a means for controlling for variability in signal intensity caused by localized variations in reagent flow rate across the microarray chip when sample and reagents are introduced to the array under conditions of laminar flow.

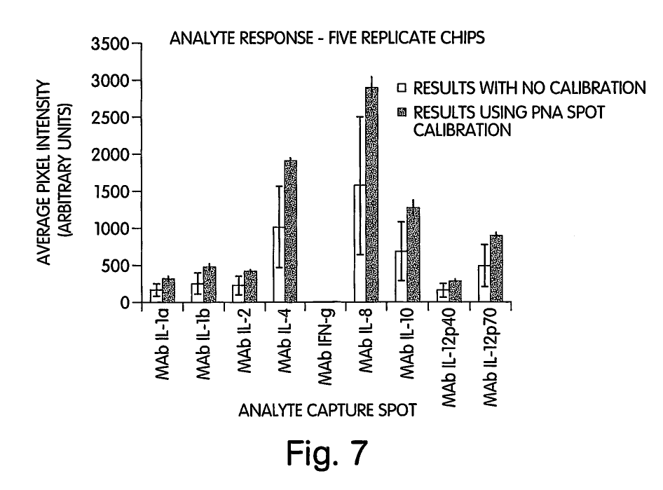

In addition, the method of the present invention may be used to reduce assay variability between similar reactions performed on different microassay chips and/or in different flow cartridges. For example, FIG. 7 shows the reduced variability across five replicate experiments that can be achieved through the use of the reference calibration oligomers described in the present application.

In addition, measuring the signal from the individual calibration spots on the array provides a means for determining that each reagent was pumped across the capture array at the appropriate flow rate, for the appropriate duration of contact with the capture spots. For quantitative analysis of multiple analytes such as antigens, the calibration spots can be used to calibrate the response levels of the separate spots, since the intensity of the calibration spots is independent of the sample source and analyte concentration.

Reagent Flow and Binding Reaction Reference Method

The microarray chip of the present invention is designed for use in a cartridge such as shown in FIG. 1. Referring to FIG. 1, the cartridge (1) includes a reaction chamber or flow cell (3) having a position for securing the microarray chip (8), and one or more reagent reservoirs (2). An enlarged view of the microchip (8) having a plurality of printed regions of interest (containing, e.g., analyte capture ligands or calibration capture ligands) in a grid-like pattern is shown as a break-out detail of FIG. 1. The reservoir or reservoirs are used to store the various samples and reagents required for conducting the binding assays that will take place on the surface of the sensor chip. The reservoirs are connected to the microarray chip cartridge reaction chamber (3) via conduits (not shown) within the cartridge that direct the flow of reagent from the reservoir(s) (2) to the chip surface (8). A separate conduit (not shown) at the opposite end of the chamber (3) from the inlet conduits leading from the reservoirs transports the sample and reagent solutions, after having flowed across the chamber and the surface of the sensor chip, to a waste or collection receptacle (7). Two or more of the reservoirs (2) may also be interconnected via conduits or inter-reservoir channels to allow for the mixing of reagents prior to contacting with the microarray chip. Fluid flow is controlled by a pressure or vacuum pump system that is removably attached to the cartridge at a pump inlet (5). Directing a reagent solution from individual reservoirs (2) to the microchip is controlled by a rotatable control valve (12) surrounded by a wall (6), the control valve includes conduits (not shown) that connect the separate channels leading from each of the reservoirs with a channel or channels leading to the cartridge reaction chamber (3). The control valve may be controlled automatically so a complete assay may be performed in an automated format. A sample injection port (4) provides an additional means for introducing a sample to the cartridge, e.g., a cartidge pre-filled with reagent and wash solutions in sealed reservoirs.

According to the present invention, each reagent reservoir that is used for a particular binding reaction will preferably include a unique calibration nucleic acid molecule, for example a peptide nucleic acid as described above, that is complementary in sequence and hybridizable with at least one calibration capture nucleic acid immobilized in a calibration reaction spot on the surface of the microarray chip. The calibration nucleic acid molecules will be non-complementary with the calibration molecules in any of the other reservoirs in order to prevent interaction between the nucleic acids of different reservoirs and, more importantly, to provide the user with the ability to monitor the reaction and flow conditions of each reagent from a particular reservoir to the surface of the microchip by detecting binding reactions between calibration nucleic acids and complementary ligands of the calibration reaction spots. However, it will be appreciated that, depending on the particular assay and components involved, not every reagent reservoir in use in the cartridge will need to include a calibration molecule. For example, if a detection molecule, such as a fluorophore in reagent, is to be included as the final step in the binding reaction, it will not be necessary to include a calibration molecule with this step of the assay as a calibration reaction spot will not react with the “naked” flurorophore.

Each of the distinct calibration nucleic acid oligomers from each reservoir that includes an oligomer, will correspond with at least one complementary calibration nucleic acid reaction spot on the chip. In this way, the microarray chip will include at least one calibration reaction spot for each reservoir that includes a calibration reaction molecule, and preferably will include at least two or more calibration reaction spots per reservoir with a calibration molecule, arranged in a columnar configuration perpendicular to the flow of reagent over the surface of the chip. The calibration reaction spots are deposited or printed on the chip at a known location or locations, the calibration reaction spot or spots being comprised of a plurality of ligands specific for a calibration nucleic acid in (preferably) only one of the reservoirs. As the contents of each reservoir, i.e., analyte, reagent, wash buffer, etc., are made to flow over the surface of the chip, monitoring the interaction between the calibration reaction spot or spots and its complementary nucleic acid from the same reservoir provides an indication of whether the conditions of the assay reaction are optimal for binding of the analyte ligand immobilized on the chip and the analyte in solution, or binding of an analyte detection ligand to the captured analyte, and calibrating the reaction.

Therefore according to the present invention, there is provided an integrated cartridge including a removable assay microarray chip and one or more reservoirs, at least one of the reservoirs including a sequentially homologous population of nucleic acids, preferably peptide nucleic acid molecules, and each of the reservoirs connected to a fluid conduit or channel for conducting reagents from the reservoirs to the microarray chip and causing the contents to flow across the chip. The cartridge further includes one or more fluid collection conduits for conducting solutions flowing across the microarray chip to one or more collection or waste receptacles.

According to one method of the present invention the binding assay and normalization/calibration steps comprise:

a. introducing a sample containing an analyte capable of binding to an analyte ligand at an analyte reaction spot immobilized on a microarray chip into one of a plurality of reservoirs and introducing an analyte detection ligand (e.g., a biotin molecule) into the same or different reservoir as the analyte, wherein the analyte detection ligand specifically binds to the analyte and is capable of providing/generating a measurable signal, such as a chemiluminescent signal, indicating that a binding reaction has occurred between the analyte and the analyte detection ligand. Preferably the analyte detection ligand is different from the analyte capture ligands;

b. introducing a unique nucleic acid calibration molecule, preferably a peptide nucleic acid, into at least one of the reservoirs that contain a reagent or sample for a particular binding assay, wherein the nucleic acid molecules in each reservoir are different, for example with respect to their nucleic acid sequence, from the homologous nucleic acid population in any of the other reservoirs and are detectable by detection means, and each nucleic acid molecule binds specifically to at least one of the calibration/nucleic acid reaction spots on the chip;

c. causing the contents of each reservoir to flow, preferably in a series (i.e., the contents of one reservoir at a time), e.g., through flow channels of a cartridge system containing the reservoirs and a reaction chamber containing a sensor chip, across the surface of a microarray chip so as to come in direct contact with one or more analyte reaction spots and one or more calibration reaction spots deposited on the sensor chip;

d. detecting the presence on the calibration reaction spot(s) of bound calibration nucleic acid molecules, the presence of one or more of these calibration molecules bound to a calibration reaction spot indicating that contact between the analyte and the analyte capture ligand has taken place and/or contact between the analyte detection ligand and the analyte has taken place.

In an alternative embodiment, the analyte, analyte detection ligand, and calibration nucleic acid molecules may all be included in one reservoir so long as there is no interaction between the components of the mixture that would interfere with the ability of the various components to bind with their intended targets on the microarray chip.

In another embodiment, any reservoir may contain more than one homologous population of calibration nucleic acid molecules to create a heterogenous population of two or more calibration nucleic acid molecules as long as the resulting heterogenous populations are non-complementary, i.e., do not hybridize or bind with each other.

Normalization/Calibration Method

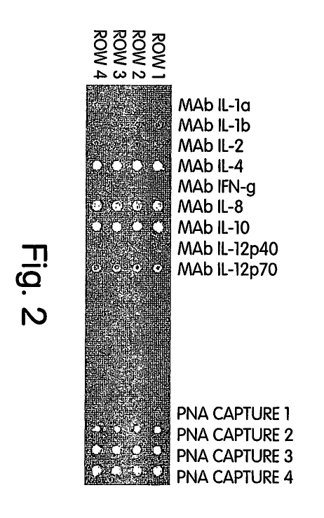

The nucleic acid reference spots, preferably peptide nucleic acids, can also be used for calibration of the array after the binding reaction has taken place. For example, the present application discloses a post-binding assay method that accounts for localized variations in flow rate of a reagent over the surface of a microarray chip where the calibration nucleic acid ligands are spotted (“calibration reaction spots”) onto the chips, preferably in one or more columns, a column being comprised of two or more reaction spots located/spotted onto the chip in such a position that the spots are in a line perpendicular to the flow of reagent over the chip. Of course, any arrangement of calibration reaction spots may be employed so long as a representative sampling of the flow characteristics across the sensor chip is achieved, but a columnar arrangement perpendicular to the direction of reagent solution flow is preferred as requiring the fewest spots to accurately sample the differential flow characteristics in different areas of the chip. Preferably each column of calibration reaction spots is deposited on the chip as a column comprising at least two homologous PNA oligonucleotide calibration reaction spots where at least one of the at least two calibration reaction spots is aligned with an analyte reaction spot, i.e., positioned in the same row, a row being comprised of at least one analyte reaction spot and at least one calibration reaction spot located/spotted onto the microarray chip such that they are aligned with each other to be parallel to the direction of flow of reagent solutions over the surface of the chip. Most preferably, a plurality of analyte capture spots and a plurality of calibration reaction spots will be arrayed in rows and columns of homologous analyte capture spots and homologous calibration reaction spots, where the columns of homologous analyte capture spots or calibration reaction spots are perpendicular to the direction of reagent flow across the chip, for example, in the manner depicted in FIG. 2.

The calibration step according to the present invention, as described below, discloses a fast, accurate, and reliable method for taking into account the variation in signal intensity between rows of a single microarray chip caused by differences in fluid flow rates within a single flow stream, over the surface of the chip from one row to the next. Subsequent to the completion of the binding reactions and washing steps, the present method comprises the following steps:

a. calculating the average pixel intensity of each calibration reaction spot and each analyte reaction spot on the chip;

b. determining the “true” average pixel intensity for each spot in step (a) by subtracting the background value, which is defined as the average pixel intensity of the area circumferentially surrounding each calibration and analyte capture spot, i.e., or any area where no binding molecules have been spotted, from the value of each spot in (a);

c. calculating a calibration factor for each array (e.g., each column) of homologous calibration reaction spots by normalizing the signals measured in step (b) for each spot in a homologous array to the replicate calibration spot in that array having the highest intensity, i.e., dividing the value of the highest intensity spot in each array (i.e., column in FIG. 2) into all the spots of lower intensity in that array of homologous spots;

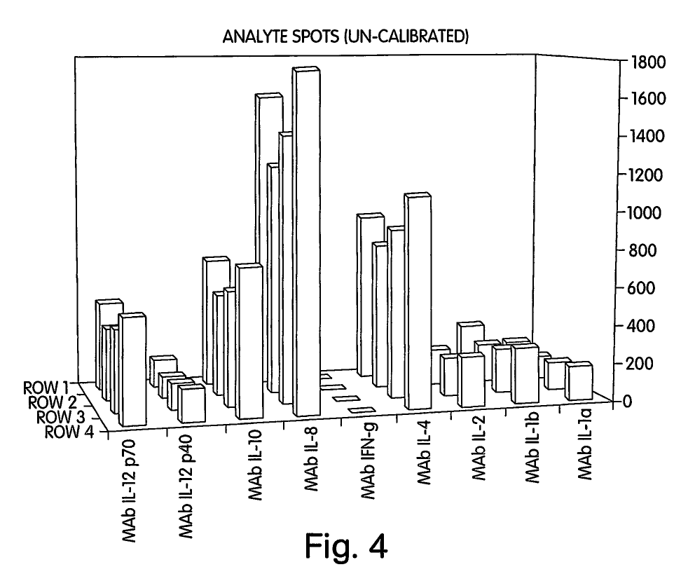

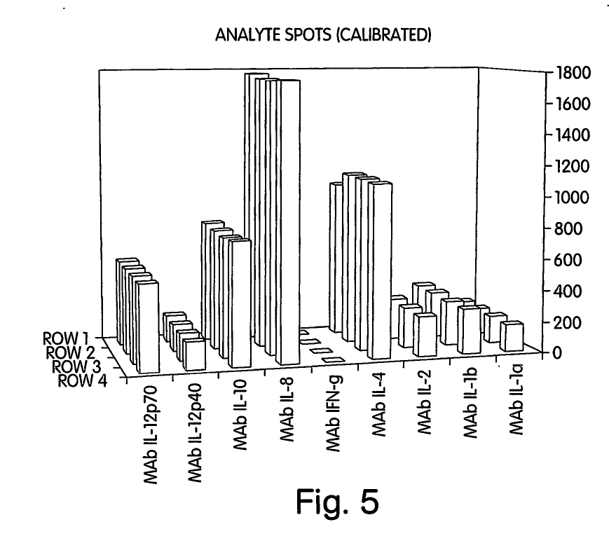

d. calculating a row-specific calibration factor by taking the average calibration value for each calibration reaction spot, i.e., the numerical result from step (c) within a row of the microarray chip, and applying that value to each analyte reaction spot in the respective row, i.e., divide the average of the row of calibration reaction spots into the value for each analyte reaction spot in the same row to get the corrected value for that row. (Compare, for example, FIG. 4 (pre-calibration) and FIG. 5 (post-calibration of the reaction shown in FIG. 4).

At a minimum, this method can be practiced with as few as two calibration reaction spots, arranged in a columnar configuration as described above, and one analyte reaction spot per chip (if deposited on the chip in the row/column pattern as described above). However, in a preferred embodiment, the calibration reaction spots are deposited as a series of columns (e.g., FIG. 2), where each column comprises at least two homologous calibration reaction spots and each separate column on the chip represents a unique set of nucleic acids, preferably PNA oligomers and the variability in flow rate of reagent over the entire surface of the chip can be accounted for and normalized. In a preferred embodiment, the assay is performed on a microchip having a combination of hundreds to thousands of analyte capture spots and calibration reactions spots per chip.

In addition, the present application discloses a post-assay calibration method to account for differences in average pixel intensity between similar or replicate binding assays or experiments carried out on more than one microarray chip in the same cartridge in simultaneous or separate experiments and/or similar reactions performed in different flow cell cartridges. This method allows for an investigator to account for differences in intensities that may occur between identical analyte reaction spots assayed on more than one microarray chip. The differences in intensity could be due to any number of factors including slight (uncontrollable) manufacturing differences between cartridges or microarray sensor chips.

Accordingly, the calibration method for comparing at least two similar binding assays performed on different microassay chips and/or in different flow cell cartridges comprises:

a. calculating the average pixel intensity of each calibration reaction spot and each analyte reaction spot on the chip as described above;

b. determining the “true” average pixel intensity for each spot in step (a) by subtracting the background value, which is defined as the average pixel intensity of the area circumferentially surrounding each calibration and analyte capture spot, i.e., or any area where no binding molecules have been spotted, from the value of each spot in (a);

c. calculating a calibration factor for each array (e.g., each column) of homologous calibration reaction spots by normalizing the signals measured in step (b) for each spot in an homologous array to the replicate calibration spot in that array having the highest intensity, i.e., dividing the value of the highest intensity spot in each array (i.e., column in FIG. 2) into all the spots of lower intensity in that array of homologous spots;

d. calculating a row-specific calibration factor by taking the average calibration value for each calibration reaction spot, i.e., the numerical result from step (c) within a row of the microarray chip, and applying that value to each analyte reaction spot in the respective row, i.e., divide the average of the row of calibration reaction spots into the value for each analyte reaction spot in the same row to get the corrected value for that row. (Compare, for example, FIGS. 4 and 5).

e. calculating a feature-specific calibration factor by normalizing the signal measured in (d) between separate chips for each homologous calibration reaction spot comprising the same calibration molecule by dividing the value of the chip with the highest intensity for each feature into the value for each corresponding feature on each of the other chip or chips;

f. calculating a chip-specific calibration factor by taking the average value for each calibration reaction spot obtained in (e) for each separate chip and applying (dividing) the chip-specific calibration factor into the signal measured for each analyte reaction spot within the array for each chip.

FIG. 7 demonstrates the reduced variability between similar assays performed on different microassay chips and in different flow cell cartridges with and without the use of the peptide nucleic acids as described in the present application. Table 3 shows the calibrated and uncalibrated values, as calculated in steps a-f above, for replicate experiments on 5 different microarray chips used to generate the graph in FIG. 7.

The present invention also contemplates a microassay chip functionalized with a plurality of analyte capture spots and calibration capture spots such as depicted in FIG. 1 (feature 8). Accordingly, the microassay chip according to the present invention includes at least one analyte reaction spot and at least one, and preferably at least two or more calibration reaction spots arranged in a column so as to be perpendicular to the flow of reagent across the surface of the chip. In a particularly preferred embodiment, the microchip of the present invention includes a plurality of calibration reaction spots arranged in a line spanning the surface of the chip. The lines of homologous spots on the microarray chip is comprised of at least two, preferably at least three, and most preferably at least 4 homologous calibration reaction spots arranged so as to span the entire breadth of the chip with respect to the direction of flow of reagent solutions introduced across the surface of the chip. Preferably the calibration reactions spots will be deposited in a line perpendicular to the flow of reagent across the surface of the chip. In a particularly preferred embodiment, the microassay chip of the present invention includes at least one, and preferably at least two or more columns of calibration reaction spots wherein each column is comprised of at least one, preferably at least two, more preferably at least three, and most preferably at least four calibration reaction spots and further wherein each column is comprised of a unique population of calibration reaction spots having ligands with a nucleic acid sequence that is substantially non-homologous with the nucleic acid sequences of the calibration reaction spots in any of the other column or columns on the chip.

The present invention also contemplates a kit comprising a pre-filled flow cell cartridge including at least one reagent and at least one functionalized microassay chip. More specifically, the pre-filled cartridge may include one or more buffers of known composition that further include a calibration molecule for use according to the present invention, said buffers may be preloaded into the reservoirs of the cartridge or provided separately. Also included in the cartridge is a pre-functionalized microchip which may be designed according to the specifications of the user and comprised of at least one and preferably at least two calibration reaction spots arranged, e.g., in a column, to span the breadth of the chip with respect to the direction of flow of reagent across the surface of the chip. Calibration reaction spots will be comprised of calibration ligands complementary to one of the calibration molecules provided in said reagent. Preferably, the microassay chip will include a plurality of analyte reaction spots and a plurality of calibration reaction spots reactive with the calibration molecules included in said buffer or buffers and wherein the calibration reaction spots are arranged in a columnar configuration so as to be perpendicular to the flow of said buffer across the surface of the chip. The pre-filled cartridge may be shrink-wrap sealed to contain the reagents and protect the reservoirs from spillage and contamination. Said kit also includes instructions for performing a microassay including the method of calibration described herein.

Instrumentation

The fluid control using air pressure to pump reagents through the cartridge channels is a feature seen in SPR commercially available cartridges, such as those manufactured by Quantech, Inc. The system allows for controlled flow rates and can include a barcode or other identification code reader which will identify both the calibration data and flow path protocol for each individual lot of cartridges.

A 12-bit or 16-bit cooled CCD camera may be used to directly image a chemiluminescent signal on the microarray. In the case of a fluorescent signal, a light source (laser, LED, or white light lamp) and appropriate filters may also be selected for use with multiple fluorescent labels.

This hardware can be arranged to interface with robotic machinery to automate the movement of cartridges between sample loading, fluid control, and imaging. As seen in FIG. 1, the flow cell cartridge includes a rotatable control valve (12) having conduits or channels (not shown) that align and connect with the reservoirs (2) on one side of the valve and with the microarray chip compartment (3) on the opposite side of the valve. The valve can be aligned manually or mechanically to direct reagent from a specific reservoir to the sensor chip.

Each of the publications cited above is incorporated herein by reference.

An example of a multiplexed binding assay using PNA reference probes is described below.

### EXAMPLES

Array Fabrication

The following cysteine-modified “capture” PNA oligomers, for use in calibration reaction spots immobilized on a microassay sensor chip according to the present invention, were designed for use as an internal reference and calibration indicator:

| | |
|---|---|
| Acetyl-Cys-OO-GTAGTCCG, | (“Capture 1”; SEQ ID NO:1) |
| Acetyl-Cys-OO-CGAAATGT, | (“Capture 2”; SEQ ID NO:2) |
| Acetyl-Cys-OO-GCGTAACT, | (“Capture 3”; SEQ ID NO:3) |
| and | |
| Acetyl-Cys-OO-TCACAAGC. | (“Capture 4”; SEQ ID NO:4) |

The following complementary biotinylated “detection” PNA oligomers, to be added to the reagent reservoirs for use as calibration reagents according to the present invention, were designed to form a duplex with the immobilized “capture” ligands on the chip:

| | |
|---|---|
| Biotinyl-OO-CGGACTAC, | (“Detection 1”; |
| | SEQ ID NO:5) |
| Biotinyl-OO-ACATTTCG, | (“Detection 2”; |
| | SEQ ID NO:6) |
| Biotinyl-OO-AGTTACGC, | (“Detection 3”; |
| | SEQ ID NO:7) |
| and | |
| Biotinyl-OO-GCT-TGT-GA. | (“Detection 4”; |
| | SEQ ID NO:8) |

In the foregoing formulae, “—OO—” represents a polyethylene glycol spacer group. All oligomers were purchased from Boston Probes, Inc. (Bedford, Mass.).

Nine anti-human cytokine matched antibody pairs, for use as capture ligands for making up the analyte reaction spots and detection ligands for recognizing “captured” cytokine analytes (OptEIA sets for capturing/detecting IL-1a, IL-1b, IL-2, IL-4, IFN-γ, IL-8, IL-10, IL-12p40, and IL-12p70) were purchased from BD Biosciences-Pharmingen (San Diego, Calif.).

Each monoclonal capture antibody was diluted to 250 μg/ml in 0.2M carbonate buffer, pH 9.0, and spotted onto a polycarbonate flow cell chip using a Packard Biochip microarray robot. Each PNA capture sequence was spotted at a concentration of 1 μM in 0.2M carbonate buffer, pH 9.0.

After spotting, the chips were immersed in a solution of 1% bovine serum albumin in PBS for 30 minutes, rinsed in water, dried and covered with a plastic top window using double-sided adhesive tape to form the flow cell gasket. Assembled flow cells were stored dry at 4° C. until use.

Reservoir reagents were prepared as follows:
1) Wash Buffer: PBS, 0.05% Tween 20, 1 nM PNA Capture 2, 5 nM PNA Capture 4;
2) Detection Antibody Mix: PBS, 0.05% Tween 20 detergent, 1 μg/ml each biotinylated detection antibody, 1 nM PNA Capture 1, 5 nM PNA Capture 3;
3) Avidin-HRP Mix: PBS, 0.05% Tween 20 detergent, 1 μg/ml Neutravidin-HRP (Pierce Chemical);
4) Luminol Reagent: SuperSignal ELISA Femto Chemiluminescent reagent (Pierce Chemical); and
5) Test Sample: PBS, 0.05% Tween 20 detergent, 1% BSA, 1 ng/ml each IL-1a, IL-1b, IL-2, IL-4, IFN-γ (IFN-g in FIGS. 2, 4), IL-8, IL-10, IL-12p40, IL-12p70.
   Cartridge Preparation and Assay Protocol

Five replicate array flow cell chips were assembled into cartridge housings. Each of the four reagent reservoirs was filled with assay reagents as follows:

| Reservoir # | Reagent |
|---|---|
| 1 | Wash Buffer |
| 2 | Detection Antibody Mix |
| 3 | Avidin-HRP Mix |
| 4 | Luminol Reagent |

The cartridge reservoirs were sealed with adhesive tape and the following reagents were contacted with the assay chip on each of four cartridges. The fifth cartridge was used as a negative control in which the Test Sample was comprised of only PBS/Tween/BSA.

| Step # | Valve Position | Flow Rate | Time |
|---|---|---|---|
| 1 | Sample | 20 μl/min | 5 min; |
| 2 | Wash Buffer | 100 μl/min | 2 min; |
| 3 | Detection Antibody Mix | 100 μl/min | 5 min; |
| 4 | Wash Buffer | 100 μl/min | 2 min; |
| 5 | Avidin-HRP | 100 μl/min | 5 min; |
| 6 | Wash Buffer | 100 μl/min | 3 min; |
| 7 | Luminol Reag. | 500 μl/min | 5 seconds. |

Immediately upon completion of the assay, an image of the cartridge flow cell was acquired using a cooled 12-bit CCD camera (Photometrics Quantix 1401E). The intensity of the chemiluminescent signal produced at each spot was measured using the QuantArray image analysis package (Packard Instrument Company).

An additional five cartridges were prepared and run using the same protocol, with the exception that no Biotinylated Detection PNA oligos were included in the reagent mixtures.

The results are shown in FIGS. 2-7. FIG. 2 shows an image of chemiluminescent signals produced by the microarray containing both capture antibody and PNA calibration spots. The variation in intensity between the capture antibody samples is a result of the different affinities of the matched antibody pairs used in the assay for each target cytokine.

FIG. 3 shows a plot of PNA calibration spot intensity for each of four rows from the microarray image shown in FIG. 2. A pattern of row-dependent signal intensity is apparent in the figure. The arrow designates the direction of laminar flow relative to the replicate calibration reaction spots. Table 1 below lists the normalization factors for each calibration reaction spot, as well as the row-specific calibration factors for the array shown in FIG. 2 as determined according to the method of the present invention.

TABLE 1
Normalization and row specific calibration factor

| | | | | | Row-Specific |
|---|---|---|---|---|---|
| | PNA Capture 1 | PNA Capture 2 | PNA Capture 3 | PNA Capture 4 | Calibration |
| | (Column 17) | (Column 18) | (Column 19) | (Column 20) | Factor |
| row 1 | 0.9091 | 0.8462 | 0.9091 | 0.9091 | 0.8934 |
| row 2 | 0.6667 | 0.6923 | 0.7273 | 0.7273 | 0.7034 |
| row 3 | 0.7576 | 0.7869 | 0.8455 | 0.8455 | 0.8089 |
| row 4 | 1.0000 | 1.0000 | 1.0000 | 1.0000 | 1.0000 |

FIG. 4 shows the intensity of the analyte capture spots for each of the ten unique capture antibodies spotted on the array. A pattern of row-dependent signal intensity is apparent in the Figure.

FIG. 5 shows the results of applying the row-specific calibration as described herein to the analyte capture spots. The variation in replicate spots is reduced as compared with FIG. 4.

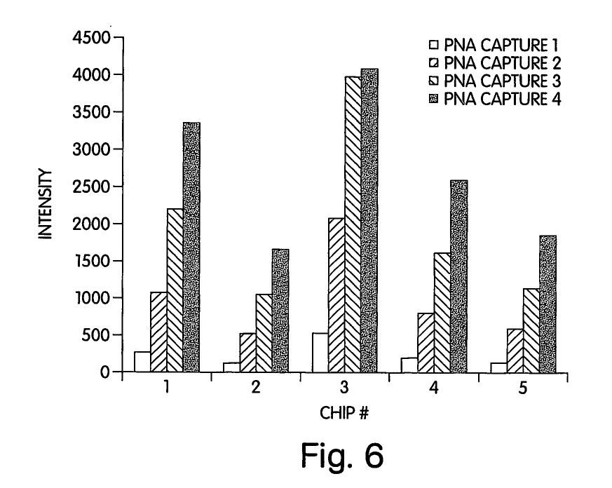

FIG. 6 displays the average signal of calibration reaction spots from five replicate chips.

Table 2 below lists the feature-specific calibration factors as well as the chip-specific calibration factors for the five replicate chips as determined according to the method of the present invention.

TABLE 2
Normalization and chip specific calibration factor

| | | | | |
|---|---|---|---|---|
| Chip 1 | Chip 2 | Chip 3 | Chip 4 | Chip 5 |

Feature-Specific Calibration Factors

| | | | | | |
|---|---|---|---|---|---|
| PNA Capture 1 | 0.4991 | 0.2377 | 1.0000 | 0.3811 | 0.2613 |
| PNA Capture 2 | 0.5151 | 0.2555 | 1.0000 | 0.3923 | 0.2912 |
| PNA Capture 3 | 0.5533 | 0.2681 | 1.0000 | 0.4103 | 0.2905 |
| PNA Capture 4 | 0.8182 | 0.4082 | 1.0000 | 0.6377 | 0.4541 |

Chip-Specific Calibration Factors

| | | | | |
|---|---|---|---|---|
| 0.5964 | 0.2924 | 1.0000 | 0.4554 | 0.3243 |

FIG. 7 shows a comparison of average response from five replicate experiments with and without the use of PNA reference spots for normalization between arrays. The graph clearly shows the reduced assay variability between arrays when the PNA reference spots and normalization method according to the present invention are employed. The calibrated and uncalibrated data in FIG. 7 is shown in Table 3.

TABLE 3
Uncalibrated and calibrated chip data

| | | | | | | |
|---|---|---|---|---|---|---|
| Chip 1 | Chip 2 | Chip 3 | Chip 4 | Chip 5 | AVG | SD |

Un-Calibrated Data

| | | | | | | | |
|---|---|---|---|---|---|---|---|
| MAb IL-1a | 175 | 95 | 312 | 137 | 121 | 168 | 85.56284 |
| MAb IL-1b | 297 | 150 | 486 | 221 | 131 | 257 | 143.755 |
| MAb IL-2 | 251 | 127 | 441 | 196 | 126 | 228.2 | 129.9296 |
| MAb IL-4 | 1116 | 576 | 1922 | 873 | 612 | 1019.8 | 549.5518 |
| MAb IFN-g | 0 | 0 | 10 | 2 | 0 | 2.4 | 4.335897 |
| MAb IL-8 | 1764 | 804 | 3101 | 1321 | 905 | 1579 | 931.6885 |
| MAb IL-10 | 788 | 389 | 1355 | 508 | 414 | 690.8 | 403.648 |
| MAb IL-12p40 | 179 | 79 | 312 | 125 | 87 | 156.4 | 95.54475 |
| MAb IL-12 p70 | 554 | 255 | 956 | 397 | 288 | 490 | 285.4514 |
| PNA Capture 1 | 275 | 131 | 551 | 210 | 144 | | |
| PNA Capture 2 | 1080.75 | 536 | 2098 | 823 | 611 | | |
| PNA Capture 3 | 2202.25 | 1067 | 3980 | 1633 | 1156 | | |
| PNA Capture 4 | 3351.25 | 1672 | 4096 | 2612 | 1860 | | |

Calibrated Data

| | | | | | | | |
|---|---|---|---|---|---|---|---|
| MAb IL-1a | 293.4111 | 324.9184 | 312 | 300.8674 | 373.1324 | 320.8659 | 31.5457 |
| MAb IL-1b | 497.9605 | 513.029 | 486 | 485.3408 | 403.9698 | 477.26 | 42.48844 |
| MAb IL-2 | 420.8353 | 434.3646 | 441 | 430.438 | 388.5511 | 423.0378 | 20.6148 |
| MAb IL-4 | 1871.124 | 1970.031 | 1922 | 1917.206 | 1887.248 | 1913.522 | 37.96916 |
| MAb IFN-g | 0 | 0 | 10 | 4.392225 | 0 | 2.878445 | 4.412041 |
| MAb IL-8 | 2957.584 | 2749.835 | 3101 | 2901.064 | 2790.784 | 2900.053 | 139.8173 |
| MAb IL-10 | 1321.188 | 1330.455 | 1355 | 1115.625 | 1276.668 | 1279.787 | 96.04318 |
| MAb IL-12p40 | 300.1176 | 270.1953 | 312 | 274.514 | 268.2853 | 285.0224 | 19.78671 |
| MAb IL-12 p70 | 928.8556 | 872.1493 | 956 | 871.8566 | 888.1169 | 903.3957 | 37.48304 |

Additional embodiments of the present invention will be apparent to those skilled in the art from considering the foregoing disclosure. All such additional embodiments are within the scope of the present invention as defined in the claims to follow.

1. A method for automatic confirming of reactions on a biosensor microarray chip, comprising:
a. providing a flow cell comprising:
i. a microarray chip having at least one analyte reaction spot and at least one calibration reaction spot deposited thereon, each analyte reaction spot comprising a plurality of analyte capture ligands specific for a particular analyte, and each calibration reaction spot comprising a plurality of calibration capture ligands for a calibration molecule different from said analyte;
ii. one or more reservoirs each including a unique calibration molecule, and each of said reservoirs connected to a fluid conduit for conducting the contents of said one or more reservoirs to the microarray chip and causing said contents to flow across said microarray chip;
iii. one or more fluid collection conduits for directing solutions flowing across the microarray chip from the microarray chip to one or more collection receptacles;
b. introducing a sample possibly containing an analyte capable of binding to said analyte reaction spot into one of said reservoirs and an analyte detection ligand into the same or different reservoir as said analyte, wherein said analyte detection ligand specifically binds said analyte and is different from said analyte capture ligands;
c. introducing a unique calibration molecule into at least one of said one or more reservoirs, wherein said calibration molecules are different from each other and are detectable by detection means, and each calibration molecule binds specifically to said calibration capture ligands immobilized on said at least one calibration reaction spot of said microarray;
d. causing the contents of each of said one or more reservoirs to flow in series across said microarray chip so as to contact said at least one analyte reaction spot and said at least one calibration reaction spot;
e. detecting the presence on said calibration reaction spots of bound calibration molecules, the presence of calibration molecules bound to a calibration reaction spot confirming that contact between said analyte and said analyte capture ligand has taken place and/or contact between said analyte detection ligand and said analyte has taken place.

2. The method of claim 1, wherein said microarray chip, said reservoirs, and said fluid conduits are in the form of an integrated cartridge.

3. The method of claim 1, wherein said analyte capture ligand and said analyte detection ligand are antibodies, Fab fragments, scFv, aptamers, nucleic acids, proteins, peptides, or other appropriate affinity molecule.

4. The method of claim 1, wherein said calibration capture ligand and said calibration molecules are nucleic acids.

5. The method of claim 4, wherein said nucleic acids are selected from the group consisting of peptide nucleic acids, DNA, and RNA.

6. The method according to claim 1, wherein said microarray chip includes at least two of said calibration reaction spots specific for one or more unique calibration reaction molecules.

7. The method according to claim 6 wherein said at least two calibration reaction spots are aligned on said microarray chip perpendicular to the flow of the contents from said reservoirs.

8. A method for calibrating a biosensor microarray chip to normalize for variations in signal intensity on said biosensor microarray chip due to localized variations in reagent flow rates over the surface of the microarray chip, said method comprising:
a. providing a flow cell comprising:
i. a microarray chip having deposited thereon at least one analyte reaction spot comprising a plurality of analyte capture ligands specific for an analyte and two or more homologous calibration reaction spots wherein each of said two or more calibration reaction spots is comprised of a plurality of calibration capture ligands specific for a calibration molecule and wherein said calibration reaction spots are deposited on said chip in a line perpendicular to the direction of reagent flow across the chip;
ii. one or more reservoirs, each connected to a fluid conduit for directing the contents of the reservoir to the microarray chip and causing said contents to flow across said microarray chip;
iii. one or more fluid collection conduits for directing solutions flowing across the microarray chip from the microarray chip to one or more collection receptacles;
b. introducing a sample possibly containing an analyte capable of binding to said analyte capture ligand into at least one of said reservoirs and an analyte detection ligand into the same or different reservoir as said analyte capture ligand, wherein said analyte detection ligand specifically binds said analyte to produce a detectable signal of measurable intensity and is different from said analyte capture ligand;
c. introducing a calibration molecule into the same reservoir as said analyte capture ligand and/or said analyte detection ligand, said calibration molecule capable of binding said calibration capture ligand to produce a detectable signal of measurable instensity;
d. causing the contents of each of said one or more reservoirs to flow in series across said microarray chip so as to contact said at least one analyte reaction spot and said two or more homologous calibration reaction spots;
e. detecting the presence on said two or more calibration reaction spots of bound calibration molecules, the presence of one or more calibration molecules bound to a calibration reaction spot indicating that contact between said analyte and said analyte capture ligand has taken place and/or contact between said analyte detection ligand and said analyte has taken place;
f. calculating the average signal intensity of each detected binding reaction on each of said calibration reaction spots and each of said analyte reaction spots;
g. calculating the background average signal intensity of an area on the surface of the chip that is not occupied by a calibration reaction spot or an analyte reaction spot and subtracting that value from said average intensity for each corresponding reaction spot calculated in step (f);
h. calculating a calibration factor for each of said two or more homologous calibration reaction spots by normalizing the values obtained in step (g) for each of said calibration reaction spots to the homologous calibration reaction spot having the highest intensity;
i. calibrating intensity values for each analyte reaction spot obtained in step (f) by dividing the intensity value for each analyte reaction spot by the calibration factor obtained in step (h).

9. The method according to claim 8, wherein said microarray chip comprises two or more columns of reaction spots deposited perpendicular to the direction of the flow of reagent solution over the surface of the chip, wherein each of said columns is comprised of two or more homologous calibration reaction spots and wherein each column of calibration reaction spots is comprised of the same or different calibration capture ligands.

10. The method according to claim 9, wherein each of said columns is comprised of unique calibration reaction spots different from the calibration reaction spots of any other columns deposited on said microarray chip.

11. The method of claim 8, wherein said microarray chip, said reservoirs, and said fluid conduits are in the form of an integrated cartridge.

12. The method of claim 8, wherein said analyte capture ligand and said analyte detection ligand are antibodies, Fab fragments, scFv, aptamers, nucleic acids, protein, peptides, or other appropriate affinity molecule.

13. The method of claim 8, wherein said calibration capture ligand and said calibration molecules are nucleic acid molecules.

14. The method of claim 13, wherein said nucleic acid molecules are selected from the group consisting of peptide nucleic acids, DNA, and RNA.

15. The method according to claim 6, wherein said one or more reservoirs include more than one population of calibration molecules and wherein said one or more population of calibration molecules are non-complementary, such that said more than one population will not form heteroduplexes within said reservoir.

16. The method according to claim 8, wherein at least two of said reservoirs include a calibration molecule, and wherein the calibration molecules in each reservoir are non-homologous to the calibration molecules in any other reservoir in said cartridge.

17. A method for calibrating a series of biosensor microarray chips to normalize for variation in signal intensity occurring between replicate binding reactions performed on two or more biosensor microarray chips;
a. providing a flow cell comprising:
i. a microarray chip having at least one analyte reaction spot and at least two homologous calibration reaction spots deposited thereon, wherein each analyte reaction spot comprises a plurality of analyte capture ligands for a particular analyte, and each calibration reaction spot comprises a plurality of calibration capture ligands different from said analyte capture ligands, and wherein binding between said at least one analyte reaction spot and said analyte or between said calibration reaction spot and a calibration molecule produces a detectable signal of measurable instensity;
ii. one or more reservoirs, each of said reservoirs connected to a fluid conduit for directing the contents of the reservoir to the microarray chip and causing said contents to flow across said microarray chip;
iii. one or more fluid collection conduits for directing solutions flowing across the microarray chip from the microarray chip to one or more collection receptacles;
b. introducing a sample possibly containing an analyte into one of said one or more reservoirs and introducing an analyte detection ligand into the same or different reservoir as said sample, wherein said analyte detection ligand specifically binds said analyte;
c. introducing a population of calibration molecules into at least one of said one or more reservoirs;
d. causing the contents of each of said reservoirs to flow in series across said microarray chip so as to contact said at least one analyte reaction spot and said at least two calibration reaction spots;
e. detecting the presence on said calibration reaction spots of bound calibration molecules, the presence of one or more calibration molecules bound to a calibration reaction spot indicating that contact between said analyte and said analyte capture ligand has taken place and/or contact between said analyte detection ligand and said analyte has taken place;
f. calculating the average pixel signal intensity of each calibration reaction spot and each analyte reaction spot on the chip;
g. calculating the background average signal intensity of an area on the surface of the chip not occupied by a calibration reaction spot or an analyte reaction spot, and subtracting that value from said average intensity for each corresponding reaction spot intensity calculated in step (f);
h. calculating a calibration factor for each homologous calibration reaction spot by normalizing the signals measured in step (g) for each homologous replicate calibration spot to that having the highest intensity, by dividing the value of the highest intensity spot into all the spots of lower intensity of homologous spots;
i. calculating a row-specific calibration factor by taking the average calibration value for each calibration reaction spot, which is the numerical result from step (h) within a row of reaction spots on the microarray chip parallel to the direction of the flow of reagent solution across the surface of the chip, and applying that value to each analyte reaction spot in the same row by dividing the average of the row of calibration reaction spots into the value for each analyte reaction spot in the same row to get the corrected value for that row.
j. calculating a feature-specific calibration factor by normalizing the signal measured in (i) between separate chips for each homologous calibration reaction spot comprising the same calibration capture ligand by dividing the value of the chip with the highest intensity for each feature into the value for each corresponding feature on each remaining chip or chips;
k. calculating a chip-specific calibration factor by taking the average value for each calibration reaction spot obtained in (O) for each separate chip and dividing the chip-specific calibration factor into the signal measured for each analyte reaction spot on the surface for each chip.

18. The method according to any one of claims 1, 8, or 17, wherein, in the detecting step, the detection ligand or the calibration molecule is detectable by measurement of the intensity of reactions selected from the group consisting of: chemiluminescence, fluorescence, colorimetry, surface plasmon resonance, electroluminescence, radiation, and MALDI-TOF mass spectra.

19. The method according to claim 17, wherein said microarray chip comprises at least two columns of said calibration reaction spots, wherein the calibration reaction spots of each column are homologous with each other and nonhomologous with the calibration reaction spots of any other column on said chip.

20. The method of claim 19, wherein said at least two columns of calibration reaction spots are deposited on said chip in an orientation perpendicular to the flow of the contents of said reservoir over the surface of said chip.

21. The method of claim 17, wherein said microarray chip, said reservoirs, and said fluid conduits are in the form of an integrated cartridge.

22. The method of claim 17, wherein said analyte capture ligand and said analyte detection ligand are antibodies, Fab fragments, scFv, aptamers, nucleic acids, proteins, peptides, or other affinity molecule.

23. The method of claim 17, wherein said calibration capture ligand and said calibration molecules are nucleic acid molecules.

24. The method of claim 23, wherein said nucleic acid molecules are selected from the group consisting of peptide nucleic acids, DNA, and RNA.

25. The method of claim 17, wherein said one or more reservoirs include more than one population of calibration molecules and wherein the calibration molecules of said more than one population of calibration molecules are non-complementary, such that said calibration molecules of said populations do not form heteroduplexes within said reservoir.

26. A microassay chip suitable for contacting reactants flowed across its surface, comprising at least one analyte reaction spot and at least two calibration reaction spots, said calibration reaction spots being positioned on said chip so as to span the breadth of the chip with respect to the direction of the flow of reactants.

27. The microassay chip of claim 26, wherein said chip includes at least three calibration reaction spots arranged in a column perpendicular to the direction of flow of reactants and wherein said calibration reaction spots are homologous.

28. The microassay chip of claim 27, wherein said chip includes at least two of said calibration reaction spot columns and wherein each column may be homologous or nonhomologous to any other calibration reaction spot column on the chip.

29. The microassay chip of claim 28, wherein said at least two columns are each comprised of nonhomologous calibration reaction spots.

30. The microassay chip of claim 26, wherein said chip includes a plurality of analyte reaction spots and wherein said analyte reaction spots may be the same or different.

31. A kit comprising a pre-filled flow cell cartridge comprising at least one reagent, said reagent comprising at least one calibration molecule, and a functionalized microassay chip disposed in said cartridge, said chip having at least two calibration reaction spots, said calibration spots being comprised of ligands specific for said at least one calibration molecule.

32. The kit according to claim 31, wherein said reagent further comprises at least one analyte.

33. The kit according to claim 32, wherein said chip includes at least one analyte reaction spot comprised of ligands specific for said analyte.


SEQUENCE LISTING (8 sequences)

SEQ ID NO: 1 - DNA, 8 bp, artificial
  DNA oligomer with Acetyl-Cys-polyethylene linker attached at the 5′ end
gtagtccg

SEQ ID NO: 2 - DNA, 8 bp, artificial
  DNA oligomer with Acetyl-Cys-polyethylene linker attached at the 5′ end
cgaaatgt

SEQ ID NO: 3 - DNA, 8 bp, artificial
  DNA oligomer with Acetyl-Cys-polyethylene linker attached at the 5′ end
gcgtaact

SEQ ID NO: 4 - DNA, 8 bp, artificial
  DNA oligomer with Acetyl-Cys-polyethylene linker attached at the 5′ end
tcacaagc

SEQ ID NO: 5 - DNA, 8 bp, artificial
  DNA oligomer with Biotinyl-polyethylene linker attached at the 5′ end
cggactac

SEQ ID NO: 6 - DNA, 8 bp, artificial
  DNA oligomer with Biotinyl-polyethylene linker attached at the 5′ end
acatttcg

SEQ ID NO: 7 - DNA, 8 bp, artificial
  DNA oligomer with Biotinyl-polyethylene linker attached at the 5′ end
agttacgc

SEQ ID NO: 8 - DNA, 8 bp, artificial
  DNA oligomer with Biotinyl-polyethylene linker attached at the 5′ end
gcttgtga